(19) United States
(12) Patent Application Publication
(10) Pub. No.: US 2009/0088833 A1
(43) Pub. Date: Apr. 2, 2009

(54) DOUBLE WALL STENT WITH RETRIEVAL MEMBER

(76) Inventor: Maximiliano Soetermans, Pinnacle, NC (US)

Correspondence Address: BRINKS HOFER GILSON & LIONE/CHICAGO/COOK, PO BOX 10395, CHICAGO, IL, 60610, US

(21) Appl. No.: 11/864,681

(22) Filed: Sep. 28, 2007

(51) Int. Cl. A61F 2/82

(52) U.S. Cl. 623/1.15

(57) ABSTRACT

A double wall stent assembly is described in which an inner stent is deployed within an outer stent. The inner stent may be attached to the outer stent by various mechanisms, including a male and female receiving end or by an adhesive. After the inner stent has become clogged, a retrieval member may be inserted into the lumen of the inner stent to detach the inner stent from the outer stent. The clogged inner stent is thereafter removed from the body lumen leaving the outer stent in place.

### TECHNICAL FIELD

The present invention relates generally to medical devices and more particularly to an inner stent coaxially disposed within an outer stent.

### BACKGROUND

Stents are used to treat occluded vessels, lumens, or organs in various physiological systems of a patient's body. For example, one or more stents are used to clear and/or open a passage through a blood vessel occlusion. As another example, stents are used to treat occlusions within the biliary system. Specifically, if a disease condition such as a tumor or an infection-related swelling causes a stenosis or other occlusion of the common bile duct, a stent may be introduced to provide an open, patent passage through the occluded region.

By way of illustration, FIG. 1A shows a plastic biliary stent 100 implanted in the common bile duct 150. The plastic biliary stent 100 provides a patent lumenal passage 110 through a stenosis 120 in the common bile duct 150. FIG. 1A also illustrates an endoscope 160 in the duodenum 152 adjacent the Ampulla of Vater 140, through which a proximal end of the biliary stent 100 extends. The endoscope 160 facilitates the placement and visual assessment of the stent 100. FIG. 1B depicts an expandable metal biliary stent 170 implanted in the common bile duct 150. Like the plastic stent 100 illustrated in FIG. 1A, the metal biliary stent 170 provides a patent lumenal passage 110 through a stenosis 120 in the common bile duct 150.

Once in place, stents (e.g., biliary stents, coronary stents) may become occluded by deposits from material passing therethrough. For example, a biliary stent may become occluded by deposits of biliary sludge (which commonly includes cholesterol crystals, calcium salts, and mucous) or microbiological organisms adhering to the interior surface of the stent. Several methods are employed to address the problems presented by the occlusion of existing (i.e., deployed) stents. Each of the methods typically includes cannulation of the stenosis or occlusion by at least a wire guide. One method is to inflate a balloon within the occluded region of the stent to compress or dislodge the occluding material and thereby re-establish at least some patency of the stent's lumenal space. Because this method is unlikely to completely remove the occluding material, re-stenosis may occur more rapidly than did the initial occlusion formation, e.g., because the occlusion already has “a foothold” to which more occluding material may be anchored and/or because the full, initial patency of the stent's internal diameter has not been re-established).

Another method is to place a second, smaller stent coaxially within the occluded stent. This method does provide a “clean,” patent lumen, but is almost certain to provide a smaller lumenal cross-section in the second, smaller stent than was present in the first stent. As a result, re-stenosis may occur more quickly than it did in the initial formation of the stenosis or occlusion.

Still another method is stent replacement. Removal and replacement of the stent provides a new, clean, open, and patent lumen. However, the procedure is time-consuming and may include increased risks if tissue around the stent has adhered to it (or, in the case of open-sided metal stents, such as expandable stents, surrounding tissue may have invaded the stent itself).

As a result, there still exists a need for an efficient, effective method and/or device for treatment of stenosis or other occlusion of the lumenal space of stents. Although the inventions described below may be useful in increasing stent patency, the claimed inventions may solve other problems as well.

### SUMMARY

Accordingly, an inner stent removably disposed within an outer stent is provided.

In a first aspect, a double wall intraluminal device is provided comprising an outer prosthesis having a first lumen and an inner prosthesis having a second lumen. The inner prosthesis is removably disposed within the outer prosthesis. One of the inner or outer prostheses comprises an engagement member. The male engagement member is adapted to removably engage the other of the inner or outer prostheses.

In a second aspect, a double wall intraluminal device is provided comprising an outer prosthesis having a first lumen and an inner prosthesis having a second lumen. The inner prosthesis is removably disposed within the outer prosthesis. One of the inner or outer prostheses comprises a cavity. The other of the inner and the outer prostheses comprises an outwardly extending protrusion adapted to removably engage with the cavity.

In a third aspect, a method for removing an inner prosthesis from an outer prosthesis within a body lumen is provided. A double wall intraluminal device is provided comprising an outer prosthesis having an outer wall surrounding a first lumen and an inner prosthesis having an inner wall surrounding a second lumen. The inner prosthesis is removably disposed within the outer prosthesis. One of the inner or outer prostheses comprises a male engagement member. The male engagement member is adapted to engage and disengage with a female receiving member located on the other of the inner or outer prostheses. A retrieval member is also provided. The retrieval member has a proximal end, an inner catheter, and an outer catheter. The proximal end is configured for positioning outside a patient's body. The inner catheter is coaxially disposed within the outer catheter. The inner and the outer catheters are movable relative to each other. The inner catheter has a cylindrical portion and a threaded distal end. The threaded distal end includes a helical thread projecting outwardly from a tapered cylindrical body portion. At least a portion of the threaded distal end is advanced into the second lumen of the inner prosthesis. The outer catheter is abutted against the outer prosthesis to substantially immobilize the outer prosthesis relative to the inner prosthesis. The proximal end of the retrieval member is then rotated to engage the threaded distal end into the second lumen of the inner wall to form an attachment between the retrieval member and the inner prosthesis. The inner prosthesis is subsequently disengaged from the outer prosthesis by retracting the inner prosthesis relative to the outer prosthesis.

The invention may include any of the above aspects in various combinations and may also include any other aspect described below in the written description or in the attached drawings.

### BRIEF DESCRIPTION OF THE DRAWINGS

Embodiments will now be described by way of example with reference to the accompanying drawings, in which:

FIG. 1A is an illustration of a plastic biliary stent disposed in a portion of biliary system;

FIG. 1B is an illustration of an expandable metal biliary stent disposed in a portion of the biliary system;

FIG. 2 is a perspective view of an inner stent disposed within an outer stent;

FIG. 3 is a plan view of an outer stent with a pin of the inner stent interlocked within a dog-legged slot of the outer stent;

FIG. 3a is a plan view of the outer stent of FIG. 3 with the inner stent interlocked into the dog-legged slot of the outer stent;

FIG. 4 is a longitudinal cross-sectional view of an inner stent with a pin element and corresponding outer stent with a dog-legged slot configured for the pin to be removably disposed thereinto;

FIG. 5 is longitudinal cross-sectional view of an outer stent with a dog-legged slot configured for the pin to be engaged thereinto;

FIG. 6 is a longitudinal cross-sectional view taken through the channel showing the inner stent locked into position within the outer stent;

FIG. 7 is an end view of the stent assembly showing the pin element of the inner stent within the channel of the outer stent;

FIG. 8 is a flat layout view of an outer stent with three dog-legged slots and an inner stent with three corresponding pins;

FIG. 9 is a perspective view of an outer stent with detents and an inner stent with corresponding indents for the detents to engage therewithin;

FIG. 10 is a end view of FIG. 9;

FIG. 11 is a perspective view of a retrieval member;

FIG. 11a is a side view of an alternative retrieval member;

FIG. 12 is cross-sectional view of the stent assembly of FIG. 8 with a retrieval member disposed within the inner stent to disengage the inner stent from the outer stent; and

FIG. 13 is a cross-sectional view of an inner stent adhered to an outer stent.

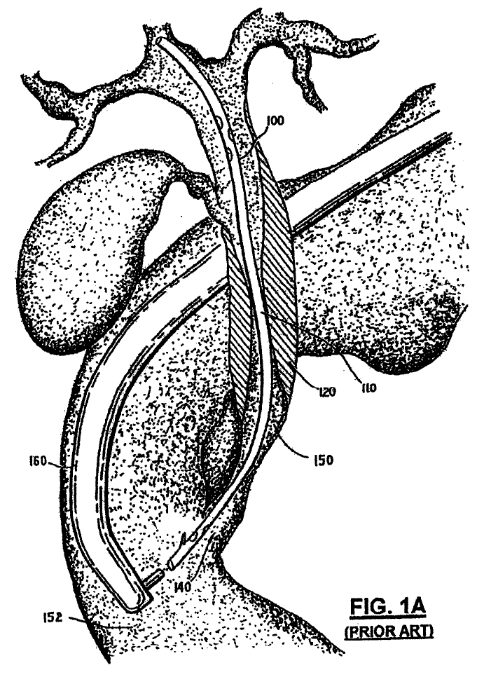

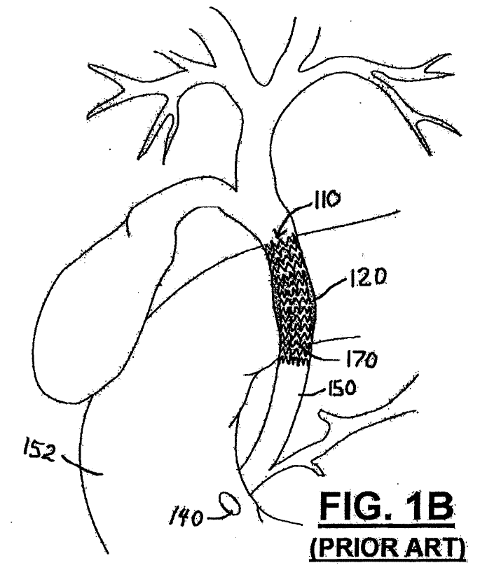

### DETAILED DESCRIPTION OF THE PREFERRED EMBODIMENTS

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

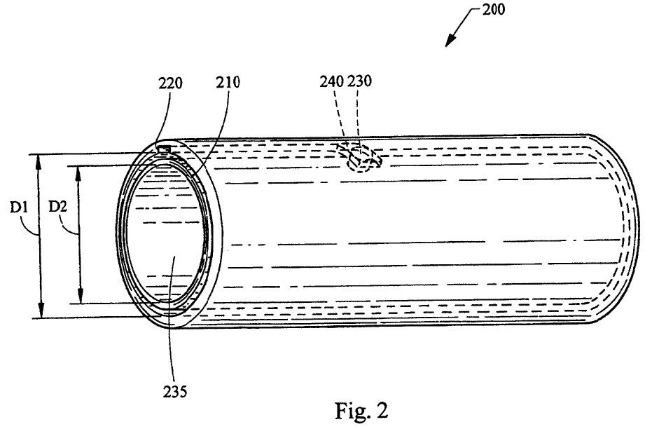

Referring now to the drawings, FIGS. 2-8 illustrate a double wall stent comprising an inner stent removably disposed within an outer stent. Preferably, the inner and outer stents are formed from a polymeric material in order to facilitate retrieval and/or replacement of the inner polymeric stent during a follow-up procedure. Generally speaking, the inner stent and outer stent are interlocked together by a pin-slot mechanism. As will be discussed below, one of the inner stent or outer stent has a pin element that removably fits into a corresponding cavity, such as a dog-legged slot, and thereafter locks into an end portion of the slot of the other stent. After the inner stent becomes clogged, a retrieval member may be inserted into the lumen of the inner stent to disengage and remove the inner stent from the outer stent.

FIG. 2 is a perspective view of a double wall stent assembly 200 including an inner stent 210 disposed within an outer stent 220. The outer stent 220 is characterized by an inner diameter of D₁ and the inner stent 210 is characterized by an inner diameter of D₂ smaller than D₁. The outer stent 220 may be deployed to a target region within a body lumen such as the biliary duct. Thereafter, the inner stent 210 is removably disposed within the outer stent 220. Alternatively, the inner stent 210 and the outer stent 220 may be deployed together into the body lumen.

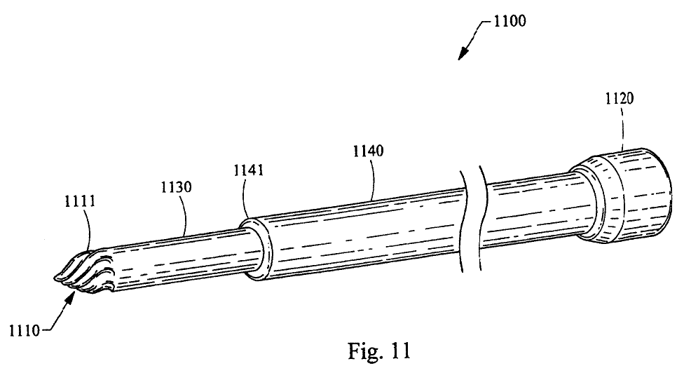

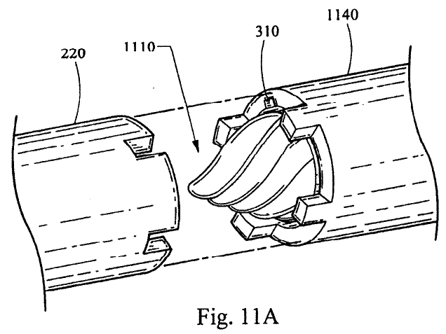

After the inner stent 210 is disposed within the outer stent 220, the inner stent 210 may engage with the outer stent 220. Specifically, the inner stent 210 has a pin element 230 that interlocks within an end portion 240 of a dog-legged slot of the outer stent 220. After patency of the inner stent 210 dissipates and it becomes occluded by deposits of biliary sludge or other matter, a retrieval member 1100 (FIG. 11) may be inserted into the lumen 235 of the inner stent 210 so as to engage the inner stent 210. A sufficient force may be applied to the retrieval member 1100 to disengage the pin element 230 from the end portion 240, thereby detaching the inner stent 210 from the outer stent 220. The inner stent 210 may be removed from the body leaving the unclogged outer stent 220 in the same implanted position as the original implanted double wall stent assembly 200 within the biliary duct. The double wall stent assembly 200 eliminates the need for recannulating into the biliary duct to deploy a replacement stent. Additionally, the outer stent 220 has a slightly larger diameter than the inner stent 210, thereby potentially prolonging the patency of the outer stent 220 relative to the inner stent 210.

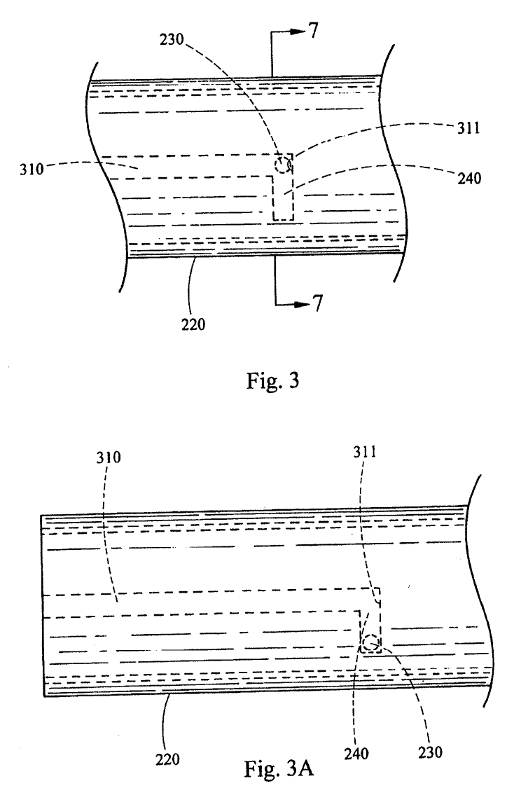

FIG. 3 is a plan view of the outer stent 220. The inner surface of the outer stent 220 includes a cavity (e.g., dog-legged slot 311) for pin element 230 of inner stent 210 to removably dispose therewithin. The dog-legged slot 311 may include a channel 310 and end portion 240. The outer stent 220 is shown with the pin element 230 of the inner stent 210 (shown in phantom) interlocked within an end portion 240 of dog-legged slot 311 of the outer stent 220. The dog-legged slot 311 has a width sufficient for the pin element 230 to removably fit thereinto. The dog-legged slot 311 may have a portion that is longitudinally aligned with the longitudinal axis of the outer stent 220 (i.e., channel 310), as shown in FIG. 3. After traveling the length of channel 310, FIG. 3A shows that the pin element 230 interlocks into the end portion 240 of the dog-legged slot 311. The interlocking of pin element 230 into the end portion 240 of the dog-legged slot 311 may be achieved by rotating the inner stent 230 a predetermined angular amount relative to the outer stent 220.

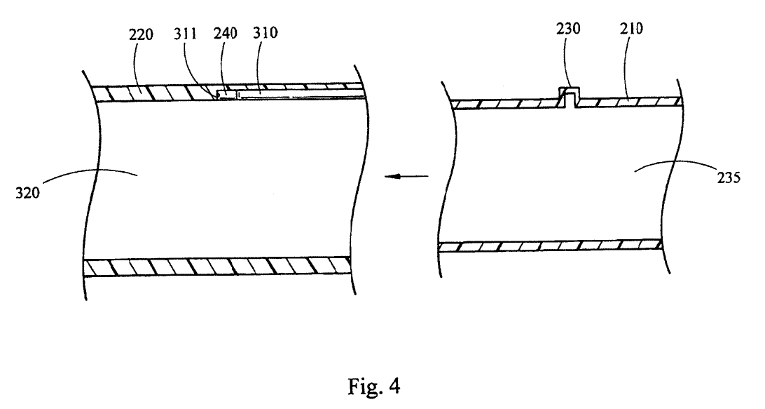

FIG. 4 is a longitudinal cross-sectional view of the inner stent 210 with a pin element 230 and a corresponding outer stent 220 with a dog-legged slot 311 for the pin element 230 to removably fit thereinto, the cross-sectional view being taken along the channel 310. FIG. 4 shows a single pin element 230 affixed to the outer wall of inner stent 210. As inner stent 210 is deployed within outer stent 220, the pin element 230 contacts the inner surface of the dog-legged slot 310. Specifically, the pin element 230 removably fits into the channel 310 to form a secure attachment with the inner surface of the outer stent 220. Lumen 320 of the outer stent 220 receives the inner stent 210.

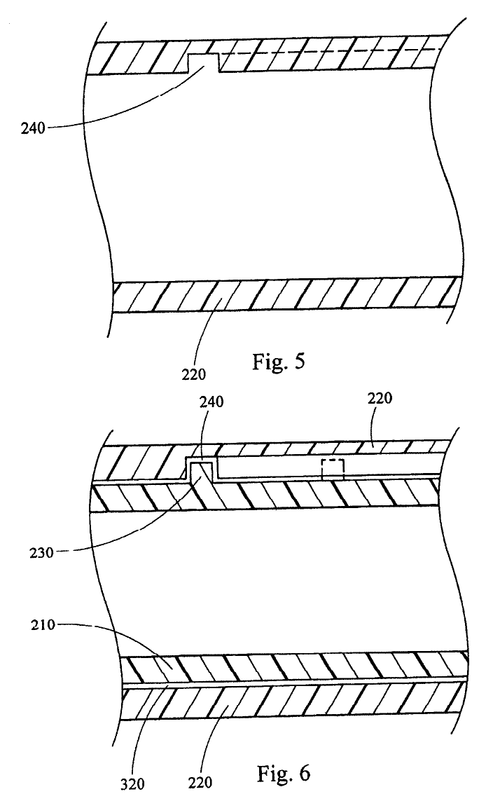

FIG. 5 is another longitudinal cross-sectional view of the outer stent 220 taken through the end portion 240 of dog-legged slot 311. The outer stent 220 is shown with the end portion 240 of dog-legged slot 311 for the pin element 230 (FIG. 6) to interlock thereinto. The end portion 240 may be sized to form a secure fit with pin element 230 such that the stent assembly 200 does not readily separate while implanted in the body lumen. At the same time, adequate clearance between the pin element 230 and end portion 240 may be provided to facilitate removal of the pin element 230 from the end portion 240.

FIG. 6 is a longitudinal cross-sectional view taken through the channel 310 of dog-legged slot 311 showing the pin element 230 of inner stent 210 locked into position within the end portion 240 of the outer stent 220. The inner stent 210 is shown as solid. FIG. 6 is the result of the pin element 230 having traveled along the entire length of the channel 310 of dog-legged slot 311, as shown by the arrow pointing in the distal direction and the dotted pin element, and subsequently rotated a predetermined angular amount to fit into the end portion 240 of dog-legged slot 311. FIG. 6 shows the inner stent 210 occupying the lumen 320 of outer stent 220. Although a single pin element 230 has been disclosed, multiple pin elements may be used to removably fit within their respective channels and slots. Furthermore, the outer stent 220 may contain the pin elements and the inner stent 210 may contain the corresponding channels and slots.

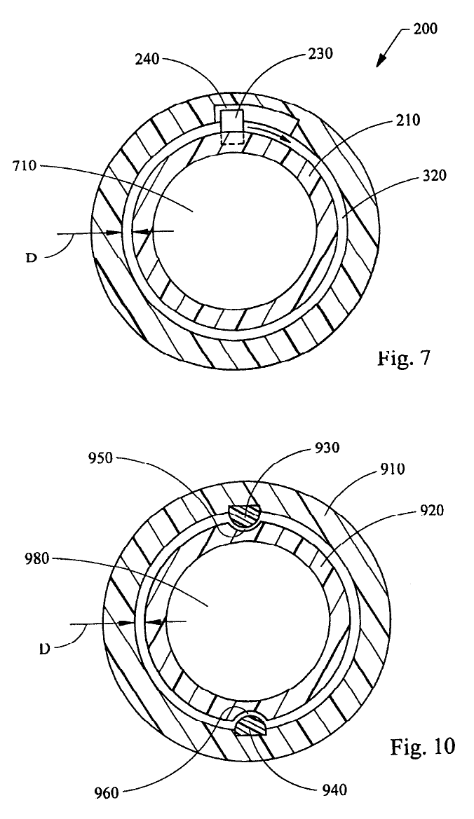

FIG. 7 is an end view of the stent assembly 200 showing the pin element 230 of the inner stent 210 within channel 310 of the outer stent 220. The clockwise arrow indicates the subsequent interlocking of the pin element 230 with the end portion 240 of dog-legged slot 311. As shown in FIG. 7, when the inner stent 210 is secured to the outer stent 220 to form the double wall stent assembly 200, bile and other matter may pass through the lumen 710 of the inner stent 210. A predetermined gap, d, between the inner and outer stents 210, 220 may exist as shown in FIG. 7 to facilitate removal of the inner stent 210 from the lumen 320 of outer stent 220. Alternatively, still referring to FIG. 7, the outer surface of the inner stent 210 and/or the inner surface of the outer stent 220 could be coated with a material to facilitate separation therebetween.

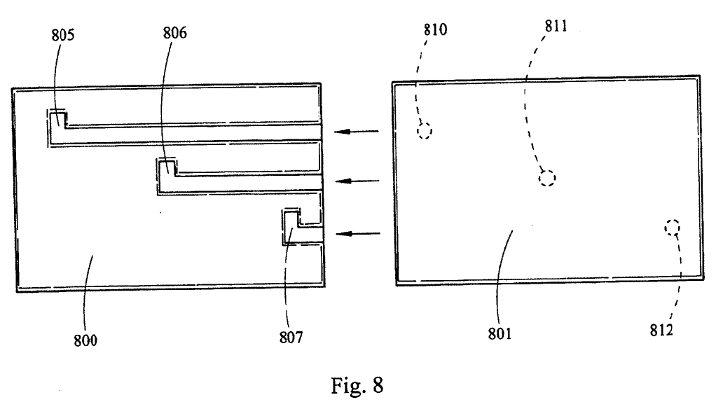

A relatively more secure fit between the inner and outer stents may be provided by having multiple pin elements engage with corresponding channels and end portions of dog-legged slots, as shown in FIG. 8. For purposes of clarity, the walls of the inner and outer stent have been illustrated as a planar surface. FIG. 8 is a flat layout view of an outer stent 800 with three channel-end portion elements 805, 806, 807 and an inner stent 801 with three corresponding pin elements 810, 811, 812. The inner stent 801 is oriented prior to insertion within outer stent 800 such that the pin elements 810, 811, 812 match up with their respective channel-end portions 805, 806, 807. As shown in FIG. 8, each of the pin elements 810, 811, 812 are configured to removably fit within their corresponding end portions 805, 806, 807, as indicated by the arrows. The pin elements 810, 811, 812 and their respective end portions 805, 806, 807 are configured such that the entire inner stent 801 is disposed within outer stent 800 when the pin elements 810, 811, 812 interlock with slots 805, 806, 807. After each of the pin elements 805, 806, 807 has traveled to the distal ends of each of their respective channel portions, the inner stent 801 is rotated a predetermined angular amount such that the pin elements 805, 806, 807 interlock into their respective end portions of the channel-end portions 805, 806, 807. The result is that inner stent 801 may be completely disposed within outer stent 800.

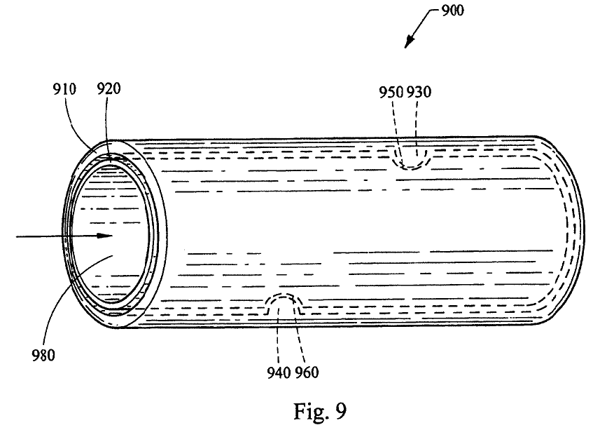

Although a pin-slot mechanism has been described, other female-male engagement mechanisms for securing the inner stent to the outer stent are contemplated. For example an outwardly extending protrusion-indent engagement mechanism is depicted in FIG. 9. FIG. 9 is a perspective view of a double wall stent assembly 900 including an outer stent 910 with outwardly extending protrusions 930, 940 (i.e., detents) and an inner stent 920 with corresponding indents 950, 960 for the detents 930, 940 to engage therewithin. The detents may be movable, compressible, or deformable. Unlike the pin-slot mechanism described above, there may be no need to rotate the inner stent 920 a predetermined angular amount in order to interlock it with the outer stent 910. Rather, the detents 930, 940 of the outer stent 910 are created along the inner surface of the outer stent 910 at predetermined positions such that they engage with the indents 950, 960 of the inner stent 920. The detent 930-indent 950 element is located distally of the detent 940-indent 960 element. Alternatively, each of the dimple-indent elements may be located at the same longitudinal position. The engagement of the detents 930, 940 with their corresponding indents 950, 960 forms a sufficiently secure connection such that the inner stent 920 does not separate from the outer stent 910. At the same time, however, a sufficient force can disengage the inner stent 920 from the outer stent 910 to withdraw the inner stent 920 from the outer stent 910. Although two detents and two indents have been shown, a single detent-indent connection can be used or more than two detent-indent connections can be used. The gap between the outer diameter of inner stent 920 and inner diameter of outer stent 910 is sufficiently small to prevent drainage of bile and other matter therethrough. Rather, bile and other matter drain into the lumen 980 of the inner stent 920, as indicated by the arrow.

FIG. 10 is an end view of FIG. 9 and illustrates the structure of the detent-indent element in greater detail. Generally speaking, the detent may be a structure with a predetermined protrusion designed to securely fit within a corresponding indent such that significant longitudinal movement of the inner stent 920 within the outer stent 910 is substantially prevented. As a result of the restriction of longitudinal movement, the inner stent 920 may be prevented from separating from the outer stent 910 while the stent assembly is implanted within the body lumen. As shown in FIG. 10, detent 930 is engaged with indent 950, and detent 940 is engaged with indent 960. The detent-indent elements are spaced apart about 180° from each other. Other detent-indent separations are possible and contemplated. For example, three detent-indent elements may be equally spaced apart around the stent assembly at about 60°. The extent to which the detents 930, 940 are protruded is dependent partly on the size of the gap, d. The protrusion of detents 930, 940 should be sufficient to create a gap, d, between the outer diameter of the inner stent 920 and the inner diameter of the outer stent 910 sufficiently small in size such that drainage through the gap is substantially eliminated.

In addition to female-male engagement mechanisms, other means for securing the inner stent to the outer stent are contemplated. FIG. 13 shows a stent assembly 1300 in which an inner stent 1330 is removably disposed and secured to an outer stent 1310 by an adhesive layer 1320. The adhesive layer 1320 forms a temporary bond between the inner stent 1330 and the outer stent 1310. Although the adhesive layer 1320 is shown to extend the entire longitudinal length of the inner and outer stents 1330, 1310 the adhesive layer may extend only a portion of the longitudinal length. Application of a sufficient force may sever the inner stent 1330 from the outer stent 1310. The outer prosthesis may comprise an outer polymeric wall and the inner prosthesis may comprise an inner polymeric wall. The inner polymeric wall of inner stent 1330 may be completely encapsulated by the outer plastic wall of outer stent 1310, as shown in FIG. 13. Because the outer wall of the inner stent 1330 is in intimate contact with the inner wall of the outer stent 1310, there is substantially no gap therebetween through which drainage may occur.

FIG. 11 is an example of a retrieval member that may be used with any of the above-described stent assemblies. FIG. 11 shows a retrieval member 1100 including a proximal end 1120 configured for positioning outside a patient's body. Furthermore, the retrieval member includes an outer catheter 1140, an inner catheter 1130 coaxially disposed within the outer catheter 1140, and a tapered threaded portion 1110 located at the distal end of the inner catheter 1130. The retrieval member provides sufficient rotational force to disengage the inner stent 210 from the outer stent 220. The inner catheter 1130 and the outer catheter 1140 are movable relative to each other. The inner catheter 1130 contains the tapered threaded portion 1110 which is capable of disengaging the pin element from the end portion 240 of a dog-legged slot 311 (FIG. 2), the detent 930 from the indent 950 (FIG. 9) or severing the temporary bond created from the inner stent 1330 adhering to the outer stent 1310 (FIG. 13). The tapered threaded portion 1110 has a helical thread 111 projecting from the tapered cylindrical body portion. The outer catheter 1140 serves as a stabilizer for the outer stent 220. In particular, as the inner catheter 1130 is rotated to disengage the inner stent from the outer stent 220, the outer stent 220 is abutted against the edge 1141 of the outer catheter 1140 for the purpose of preventing the outer stent 220 from moving from its target implanted site as the inner stent 210 is pulled out from the within the lumen of the outer stent 220.

Although only a single inner stent has been described coaxially disposed within an outer stent in the above stent assemblies, more than one inner stent may be disposed within the outer stent.

Having described examples of various stent assemblies, a method of implanting and removing the inner stent from within the outer stent will now be described. After the outer stent 220 has been deployed in a target body lumen, such as a biliary duct, the inner stent 210 can be deployed into the lumen 320 of the outer stent 220 (FIG. 4). Alternatively, the inner stent 210 and the outer stent 220 may be deployed together into the target body lumen. Delivery and deployment of the inner stent 210 and the outer stent 220 may be achieved using a standard push-pull delivery catheter, such as the Oasis® deployment device manufactured and sold by COOK®.

After the inner stent 210 has been disposed within the outer stent 220, the inner stent 210 may engage with the outer stent 220. As the inner stent 210 engages the lumen 320 of the outer stent 220, the pin element 230 of the inner stent 210 removably fits within the channel 310 of the outer stent 220, as shown in FIGS. 3, 6, 7 and 8. The pin element 230 continues to removably move distally within channel 310. The pin element 230 stops moving distally when it contacts the distal edge of the channel 310 (FIG. 4). The locations of the distal edge of the channel 310 and the pin element 230 are configured such that the inner stent 210 is completely disposed within the lumen 320 of the outer stent 220 when the pin element 230 has reached the distal edge of the channel 310. When the pin element 230 has reached the distal edge of the channel 310, the proximal end of the delivery catheter is torqued to rotate the inner stent 210 a predetermined angular amount to lock the pin element 230 into the end portion 240 of dog-legged slot 311, as shown in FIG. 7. The locking of the pin element 230 into end portion 240 may be sufficient to substantially restrict longitudinal movement of the inner stent 210 relative to the outer stent 220, thereby preventing separation of the inner stent 210 from the outer stent 220. The locking of the pin element 230 into the end portion 240 is shown in FIG. 7. FIG. 6 shows that the locking mechanism enables the inner stent 210 to be securely disposed within the lumen 320 of the outer stent 220.

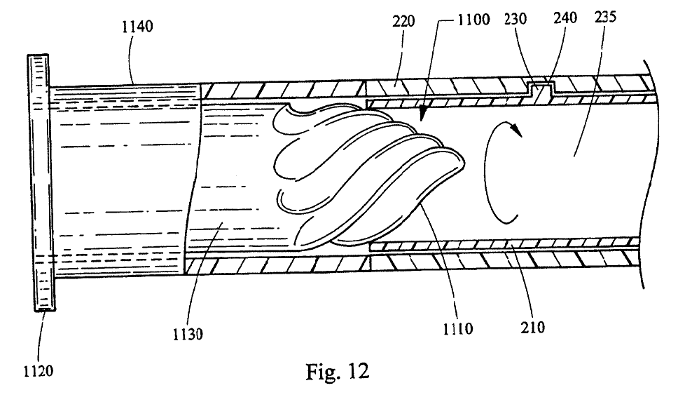

The fixed diameter of the inner diameter of the inner stent 210 will typically become clogged in the biliary duct within three months or so. When the inner stent 210 becomes clogged, a retrieval member, such as the retrieval member 1100 of FIG. 11, may be used to disengage the inner stent 210 from the outer stent 220 and thereafter withdraw the inner stent 210 from the biliary duct, as will be now explained with reference to FIG. 12. The retrieval member 1100 may be advanced such that the threaded distal end 1110 is positioned within the lumen 235 of the inner stent 210 and the outer catheter 1140 is abutted against the outer stent 220 to substantially immobilize the outer stent 220 during removal of the inner stent 210 to substantially prevent rotational and longitudinal movement of the outer stent 220 relative to the inner stent 210. Alternatively, as shown in FIG. 11a, the outer catheter 1140 may have one or more engagement members 1145 which engages one or more receiving members 1146 of the outer stent 220 to substantially prevent rotational and longitudinal movement therebetween during removal of the inner stent 210 from the outer stent 220.

The threaded distal end 1110 is positioned such that it engages the end of the inner stent 210, as shown in FIG. 12. With the outer catheter 1140 substantially immobilized by the outer stent 220, the proximal end 1120 of the retrieval member 1100 may be rotated. Rotation of the proximal end 1120 of the retrieval member 1100 causes the tapered threaded distal end 1110 to threadably engage with the inner surface of the inner stent 210 such that the inner stent 210 disengages from the outer stent 220. The inner stent 210 may then be pulled out of the lumen 320 of the outer stent 220 leaving in place the outer stent 220 within the desired region of the biliary duct. The above-described procedure eliminates the need to recannulate into the biliary duct to deploy a replacement stent. Additionally, the outer stent 220 has a slightly larger diameter than the inner stent 210, thereby potentially prolonging the patency of the outer stent 220 relative to the inner stent 210.

Although the above procedure of deploying and withdrawing an inner stent from the outer stent has been described with respect to an inner stent attached by a pin-channel mechanism to the outer stent, the identical procedure may also be applied to an inner stent attached to the outer stent by a detent-indent mechanism (FIGS. 9-10) or any other type of male-female connection. Furthermore, the above-described procedure may also be used to remove an inner stent that is adhered to the outer stent (FIG. 13). Additionally, the above-described procedure may be used to implant and withdraw multiple inner stents from an outer stent.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A double wall intraluminal device comprising:
an outer prosthesis having a first lumen;
an inner prosthesis having a second lumen, the inner prosthesis removably disposed within the outer prosthesis, wherein one of the inner or outer prostheses comprises an engagement member, the engagement member adapted to removably engage the other of the inner or outer prostheses.

2. The double wall intraluminal device of claim 1, wherein the inner prosthesis is removable from the outer prosthesis by the application of a predetermined force to the inner prosthesis relative to the outer prosthesis sufficient to disengage the engagement member from the other of the inner or outer prosthesis.

3. The double wall intraluminal device of claim 1, wherein the other of the inner or outer prosthesis comprises a receiving member removably engaged by the engagement member.

4. The double wall intraluminal device of claim 3, wherein the engagement member comprises a detent structure and the receiving member comprises an indent adapted to receive the detent structure, the detent structure being one of movable, compressible, or deformable.

5. The double wall intraluminal device of claim 3, wherein the engagement member comprises a pin element and the receiving remember comprises a dog-legged slot.

6. The double wall intraluminal device of claim 1, wherein the engagement member comprises an adhesive disposed between the inner and the outer prostheses to form a temporary bond therebetween.

7. The double wall intraluminal device of claim 1, wherein one of the inner or outer prostheses comprises a plurality of engagement members adapted to engage and disengage with a plurality of corresponding receiving members on the other of the inner or outer prosthesis.

8. The double wall intraluminal device of claim 7, wherein the plurality of engagement members and the plurality of corresponding receiving members are circumferentially disposed about the inner and outer prostheses.

9. The double wall intraluminal device of claim 3, wherein a retrieving member provides the predetermined force, the retrieving member having a threaded distal end to threadably engage the inner prosthesis.

10. The double wall intraluminal device of claim 9, wherein the threaded distal end includes a helical thread projecting from a tapered cylindrical body portion, the threaded distal end threadably engaging the inner prosthesis to form an attachment between the retrieval member and the inner prosthesis.

11. The double wall intraluminal device of claim 10, wherein at least a portion of the helical thread is positioned within the second lumen of the inner prosthesis.

12. The double wall intraluminal device of claim 11, wherein the retrieving member comprises an inner catheter coaxially disposed within an outer catheter, the inner and outer catheters being movable relative to each other, the threaded distal end being affixed to the inner catheter.

13. The double wall intraluminal device of claim 12, wherein the outer catheter of the retrieval member substantially immobilizes the outer prosthesis as the inner catheter of the retrieval member disengages the inner prosthesis from the outer prosthesis.

14. A double wall intraluminal device comprising:
an outer prosthesis having a first lumen;
an inner prosthesis having a second lumen, the inner prosthesis removably disposed within the outer prosthesis, wherein one of the inner or outer prostheses comprises a cavity, the other of the inner and the outer prostheses comprising an outwardly extending protrusion adapted to removably engage with the cavity.

15. The double wall intraluminal device of claim 14, wherein the cavity comprises a dog-legged slot.

16. The double wall intraluminal device of claim 14, wherein the outwardly extending protrusion comprises a pin element.

17. The double wall intraluminal device of claim 14, wherein the outwardly extending protrusion comprises a detent, the detent being one of movable, compressible, or deformable.

18. A method for removing an inner prosthesis from an outer prosthesis, comprising the steps of:
(a) providing a double wall intraluminal device comprising:
an outer prosthesis having an outer wall surrounding a first lumen; and
an inner prosthesis having an inner wall surrounding a second lumen, the inner prosthesis removably disposed within the outer prosthesis, wherein one of the inner or outer prostheses comprises a male engagement member adapted to engage and disengage with a female receiving member located on the other of the inner or outer prosthesis;
(b) providing a retrieval member comprising:
a proximal end configured for positioning outside a patient's body;
an inner catheter coaxially disposed within an outer catheter, the inner and the outer catheters being movable relative to each other, the inner catheter having a cylindrical portion and a threaded distal end, wherein the threaded distal end includes a helical thread projecting from a tapered cylindrical body portion;
(c) advancing at least a portion of the threaded distal end into the second lumen of the inner prosthesis;
(d) abutting the outer catheter against the outer prosthesis to substantially immobilize the outer prosthesis relative to the inner prosthesis;
(e) rotating the proximal end of the retrieval member to engage the threaded distal end with the inner wall of the inner prosthesis to form an attachment between the retrieval member and the inner prosthesis; and
(f) disengaging the inner prosthesis from the outer prosthesis.

19. The method of claim 18, further comprising the step of:
(g) retracting the inner catheter from within the first lumen of the outer prosthesis.

20. The method of claim 19, wherein step (f) further comprises retracting the inner prosthesis relative to the outer prosthesis.

21. The method of claim 18, wherein step (c) further comprises disposing the double wall intraluminal device within a bodily lumen of a patient.

22. The method of claim 18, wherein step (f) further comprises removing the double wall intraluminal device from a bodily lumen of a patient.

23. A double wall intraluminal device comprising:
an outer prosthesis having a first lumen;
an inner prosthesis having a second lumen, the inner prosthesis removably disposed within the outer prosthesis, wherein one of the inner or outer prostheses comprises a dog-legged slot, the other of the inner and the outer prostheses comprising an outwardly extending protrusion adapted to removably engage with the cavity, the outwardly extending protrusion comprising a pin element or a detent.
